(19) United States
(12) Patent Application Publication
(10) Pub. No.: US 2009/0081761 A1
(43) Pub. Date: Mar. 26, 2009

(54) Modified hydrogenase, enzymatic electrode made of modified hydrogenase, and hydrogenase modification method

(76) Inventors: Hidetaka Nishikoori, Susono-shi (JP); Hirofumi Nishihara, Ushiku-shi (JP); Ki-Seok Yoon, Inashiki-gun (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/213,081

(22) Filed: Jun. 13, 2008

(30) Foreign Application Priority Data
Jun. 14, 2007 (JP) 2007-157687

(51) Int. Cl. C12N 9/14

(52) U.S. Cl. 435/195

(57) ABSTRACT

The invention provides: (1) a modified hydrogenase obtained by removing electron-transfer sites from a hydrogenase constituted of: active subunits including active sites having a hydrogen oxidization-reduction activity; and electron-transfer subunits having the electron-transfer sites through which electrons are transferred between the active sites and the outside of the hydrogenase; (2) a modified hydrogenase obtained from a hydrogenase, wherein when the hydrogenase is isolated from bacteria that produces the hydrogenase, a process for exposing the hydrogenase to an oxygen atmosphere is executed; (3) an enzymatic electrode made of at least one of the foregoing modified hydrogenases; and (4) a hydrogenase modification method including: a step of isolating from hydrogenase-producing bacteria; and a step of removing the electron-transfer sites of the electron-transfer subunits from the hydrogenase by exposing the hydrogenase to an oxygen atmosphere.

### INCORPORATION BY REFERENCE

The disclosure of Japanese Patent Application No. 2007-157687 filed on Jun. 14, 2007 including the specification, drawings and abstract is incorporated herein by reference in its entirety.

### BACKGROUND OF THE INVENTION

1. Field of the Invention

The invention relates to modified hydrogenases, enzymatic electrodes made of modified hydrogenases, and hydrogenase modification methods.

2. Description of the Related Art

Fuel cells directly convert chemical energy into electric energy through electrochemical oxidization of fuel between two electrically-connected electrodes to which fuel and oxidant are supplied, respectively. Unlike thermal power generation, fuel cells are not restricted by the Carnot cycle, and thus they provide a high energy conversion efficiency. In particular, solid polymer electrolyte fuel cells using solid polymer electrolyte membranes can be easily made compact in size and also they can operate at a low temperature. Having such advantages, solid polymer electrolyte fuel cells have been attracting much attention as power sources for mobile phones and vehicles, especially.

The reaction represented by the formula (1) shown below occurs at the anode of a solid polymer electrolyte fuel cell.

H₂→2H⁺+2e⁻ (1)

The electrons produced from the reaction of the formula (1) travel to external loads via external circuits and work at the external loads, and then they reach the cathode (oxidant electrode). The protons produced from the reaction of the formula (1) move in the solid polymer electrolyte fuel cell from the anode side to the cathode side due to electroosmosis.

On the other hand, the reaction represented by the formula (2) shown below occurs at the cathode.

4H⁺O₂+4e⁻ [symbol] 2H₂O (2)

As electrodes for facilitating the reactions of the formulas (1) and (2), platinum and platinum alloys have been typically used due to their high catalytic activities. However, because platinum is very precious and therefore very expensive, various new electrode catalysts have been under development as substitutes for platinum and platinum alloys.

Among such new electrode catalysts, hydrogenases (hydrogen oxidization-reduction enzymes) have been attracting much attention. Because hydrogenases are derived from organisms, they can be mass-produced by culturing. Further, it is said that the hydrogen oxidization activity of hydrogenases is as strong as or even stronger than that of platinum, and also the reactivity of hydrogenases is strong even at a room temperature. For example, it is described in page 589-591 of “Solid state communications, vol. 133, No. 9 (2005)”, which is a non-patent reference, that hydrogenases can be controlled to provide a catalytic activity as strong as that of platinum catalyst.

Hydrogenases have been continuously studied and researched and various technologies for utilizing their catalytic effects have been proposed (Refer to Solid state communications, vol. 133, No. 9, page 589-591 (2005), Japanese Patent Application Publications No. 2005-501387 (JP-A-2005-501387), No 2000-350585 (JP-A-2000-350585), No. 04-365474 (JP-A-04-365474), and No. 2002-214190 (JP-A-2002-214190), etc.). Hydrogenases are enzymes that can serve as hydrogen oxidization reduction catalysts, and they are normally proteins constituted of (1) active subunits (large subunits) located in the three-dimensional structures of the hydrogenases and serving as hydrogen oxidization reduction catalysts (having a hydrogen oxidization activity and a hydrogen producing activity) and (2) electron-transfer subunits (small subunits) containing electron-transfer sites through which the electrons produced from oxidization of hydrogen molecules and the electrons needed for producing hydrogen are transferred between the outside of the hydrogenase and the active sites.

However, because hydrogenases are unstable enzymes, their catalytic effects are deteriorated by oxygen, which may lead to loss of their activities. Therefore, to enable more practical researches and practical use of hydrogenases, it is important to obtain hydrogenases more stable against external factors and less restrictive for use conditions.

Thus, various researches have been conducted to obtain more stable hydrogenases. For example, Japanese Patent Application Publication No. 2000-350585 (JP-A-2000-350585) describes high heat-resistant and high oxygen-resistant hydrogenase proteins that are obtained through modification of amino-acid sequences for increasing their heat resistance and oxygen resistance. Further, Japanese Patent Application Publication No. 04-365474 (JP-A-04-365474) describes hydrogen bacteria obtained from acidophilic thermophilic bacteria or acidophilic mesophilic bacteria and having hydrogenases that can maintain their activities even under an oxidative condition, and it also describes hydrogenases obtained from such hydrogen bacteria.

### SUMMARY OF THE INVENTION

The invention provides a modified hydrogenase having a high oxygen resistance and a high stability, and an enzymatic electrode made of a modified hydrogenase having a high oxygen resistance and thus capable of maintaining stable electrode characteristics for a long period of time. Further, the invention provides a hydrogenase modification method for obtaining modified hydrogenases having a high oxygen resistance.

The first aspect of the invention relates to a modified hydrogenase obtained by removing electron-transfer sites from a hydrogenase constituted of: active subunits including active sites having a hydrogen oxidization reduction activity; and electron-transfer subunits having the electron-transfer sites through which electrons are transferred between the active sites and the outside of the hydrogenase.

The above-described modified hydrogenase may be such that the removal of the electron-transfer sites is accomplished by removing the electron-transfer subunits. Further, the above-described modified hydrogenase may be such that the hydrogenase is a [Ni—Fe] hydrogenase constituted of active subunits having active sites containing Ni—Fe and electron-transfer subunits having electron-transfer sites containing Fe—S clusters.

The above-described hydrogenase may be a modified hydrogenase obtained from a hydrogenase constituted of: active subunits including active sites having a hydrogen oxidization reduction activity; and electron-transfer subunits having electron-transfer sites through which electrons are transferred between the active sites and the outside of the hydrogenase. According to this modified hydrogenase, when the hydrogenase is isolated from bacteria that produces the hydrogenase, a process for exposing the hydrogenase to an oxygen atmosphere is executed.

In the above case, considering the fact that the oxygen resistance of the electron-transfer sites is low, the electron-transfer sites are separated from the hydrogenase by exposing the hydrogenase to an oxygen atmosphere, whereby a modified hydrogenase free of the electron-transfer sites is obtained. The process for exposing the hydrogenase to the oxygen atmosphere may be a process in which membrane fractions are isolated from the hydrogenase-producing bacteria, the isolated membrane fractions are exposed to an oxygen atmosphere, and then the hydrogenase is solubilized from the membrane fractions or a process in which membrane fractions are isolated from the hydrogenase-producing bacteria and the hydrogenase is solubilized from the membrane fractions under an oxygen atmosphere.

The source of the hydrogenase is not limited specifically. For example, the hydrogenase may be a hydrogenase originated from Hydrogenovibrio marinus.

As such, the invention provides a modified hydrogenase having a high oxygen resistance and capable of maintaining its hydrogen oxidization reduction activity (a hydrogen oxidization activity or a hydrogen production activity) for a long period of time even in the presence of oxygen.

The second aspect of the invention relates to an enzymatic electrode made of the modified hydrogenase according to the first aspect of the invention. This enzymatic electrode can maintain stable electrode characteristics for a long period of time.

The third aspect of the invention relates to a hydrogenase modification method. This method includes: isolating from hydrogenase-producing bacteria a hydrogenase constituted of: active subunits including active sites having a hydrogen oxidization reduction activity; and electron-transfer subunits having electron-transfer sites through which electrons are transferred between the active sites and the outside of the hydrogenase; removing the electron-transfer sites of the electron-transfer subunits from the hydrogenase by exposing the hydrogenase to an oxygen atmosphere.

In the above case, considering the fact that the oxygen resistance of the electron-transfer sites is low, the electron-transfer sites are separated from the hydrogenase by exposing it to an oxygen atmosphere. As such, the oxygen resistance of the hydrogenase can be easily increased without amino-acid mutation and the like.

The above-described method may include a step of isolating membrane fractions from the hydrogenase-producing bacteria, a step of exposing the isolated membrane fractions to the oxygen atmosphere, and a step of solubilizing hydrogenase from the membrane fractions after the exposure to the oxygen atmosphere. Alternatively, the above-described method may include a step of isolating membrane fractions from the hydrogenase-producing bacteria and a step of solubilizing the hydrogenase from the isolated membrane fractions under an oxygen atmosphere.

Thus, each modified hydrogenase of the invention has a high oxygen resistance and is capable of maintaining its stable catalytic activity for a long period of time. Thus, it is possible to provide enzymatic electrodes more durable and less restrictive for use conditions. Further, the modified hydrogenases of the invention can be produced in very simple manners, and therefore their productivity is high.

### BRIEF DESCRIPTION OF THE DRAWINGS

The foregoing and further objects, features and advantages of the invention will become apparent from the following description of embodiments with reference to the accompanying drawings, wherein like numerals are used to represent like elements and wherein:

FIG. 1A and FIG. 1B are views conceptually showing modified hydrogenases according to the invention and intact-type hydrogenates according to a related art; and

FIG. 2A and FIG. 2B are charts representing the results of the activity measurement and electrophoresis conducted to modified hydrogenases of an example of the invention.

### DETAILED DESCRIPTION OF THE EMBODIMENTS

Hereinafter, an example of the invention will be described which relates to hydrogenases constituted of (1) active subunits containing active sites having a hydrogen oxidization-reduction activity and (2) electron-transfer subunits including electron-transfer sites through which electrons are transferred between the active sites and the outside of the hydrogenases. In this example of the invention, modified hydrogenases are obtained by removing the electron-transfer sites of the electron-transfer subunits.

Typically, hydrogenases have catalytic activities for hydrogen oxidization reactions and for hydrogen production reactions. Hydrogenases are constituted of (1) active subunits containing active sites directly related to hydrogen oxidization and hydrogen production and (2) electron-transfer subunits containing electron-transfer sites through which the electrons produced from hydrogen oxidization reactions are transferred from the active sites to the outside of the hydrogenase and through which the electrons necessary for hydrogen production reactions are transferred from the outside of the hydrogenases to the active sites.

As mentioned above, hydrogenases have significant activities as hydrogen oxidization catalysts or as hydrogen production catalysts. However, having a low oxygen resistance, the functional stability of hydrogenases is low under an oxygen atmosphere, and thus it is difficult to keep its catalytic activity stable for a long period of time. In view of this, the present inventors have studied hydrogenases carefully and discovered that, among the active subunits containing the active sites that have a great influence on the hydrogen oxidization-reduction activities (hydrogen oxidization activities or hydrogen production activities) of hydrogenases and the electron-transfer subunits including the electron-transfer sites, the oxygen resistance of the electron-transfer sites is particularly low and thus they tends to be functionally damaged by oxygen.

If the electron-transfer functions of the electron-transfer sites through which electrons are transferred between the outside of the hydrogenase and the active sites of the hydrogenase are lost due to their functional deterioration or due to their degradation and separation caused by external factors (e.g., oxidization), the electrons produced at the active sites become unable to be transferred to the outside of the hydrogenase and the electrons necessary for producing hydrogen become unable to be transferred to the active sites from the outside of the hydrogenase.

In this example of the invention, an experiment was conducted in which electron-transfer sites (typically, electron-transfer subunits containing electron-transfer sites) were artificially removed from intact-type hydrogenases (dimmer-form hydrogenases constituted of active subunits and electron-transfer subunits), so that the oxygen resistance of the hydrogenases increased significantly.

If electron-transfer sites are removed from hydrogenases in advance, functional deterioration and decomposition/separation of the hydrogenases due to oxidization do not occur even under an oxygen atmosphere. Therefore, even if the hydrogenases are exposed to an oxygen atmosphere for a long period of time, they maintain stable hydrogen oxidization activities and hydrogen production activities. If electron-transfer sites are not removed in advance unlike the case of the above-described modified hydrogenases of the example of the invention, presumably, the electron-transfer sites of the hydrogenases are decomposed and separated as they continues to be exposed to an oxygen atmosphere during use, and the functionally deteriorated, decomposed, or separated electron-transfer sites inhibit transfer of electrons between the active sites of the hydrogenases and the outside of the hydrogenases, and as a result, the hydrogen oxidizing producing ability and the hydrogen producing ability of the hydrogenases decrease down to levels lower than those of the foregoing modified hydrogenases from which the electron-transfer sites have been removed in advance. However, note that in some cases the initial catalytic activity of the modified hydrogenases of the example of the invention is weaker than that of intact-type hydrogenases.

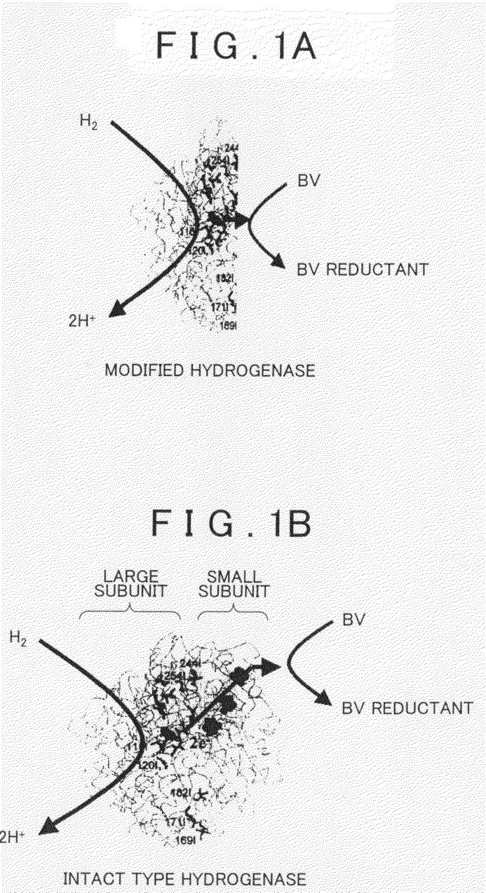

Typically, the modified hydrogenase of the example of the invention is constituted only of active subunits after removal of electron-transfer subunits. Presumably, the modified hydrogenases constituted only of the active subunits allows direct transfer of electrons between the active sites and the outside of the hydrogenase. Normally, the active sites of intact-type hydrogenase are located near the center of the three-dimensional structure of a hydrogenase protein, and the electron-transfer sites through which electrons are transferred between the active sites in the three-dimensional structure and the outside of the hydrogenase. Therefore, if the electron-transfer subunits are removed, the positions of the active sites become closer to the surfaces of the three-dimensional structure, which allows direct electron transfer between the active sites and the outside of the hydrogenase (Refer to FIG. 1A).

In this example of the invention, as long as the electron-transfer sites can be removed from the hydrogenase, the entire electron subunits containing the electron-transfer sites are not necessarily removed. That is, constituents of the electron-transfer subunits other than the electron-transfer sites may be left in the hydrogenase. However, typically, all the electron-transfer sites contained in the hydrogenase are removed by removing the entire electron-transfer subunits.

The hydrogenases (intact type) cited in this example of the invention are not limited specifically, and their sources are not limited specifically neither. The following are examples of hydrogenase sources: Hydrogenovibrio bacteria (e.g., Hydrogenovibrio marinus); Desulfovibrio bacteria (e.g., Desulfovibrio vulgaris, Desulfovibrio gigas); and Clostridium bacteria (e.g., Clostridium pasteurianum).

The following are other examples of hydrogenase sources: Hydrogenophaga bacteria (e.g., Hydrogenophaga sp.), Pyrodictium brockii; Thermotoga maritima, Bacillus schlegelii; and Clostridium thermoaceticum.

The following are other examples of hydrogenase sources: Ralstonia bacteria (e.g., Ralstonia eutropha), Thiocapsa bacteria (e.g., Thiocapsa roseopersicina); Oligotropha carboxidovorans; Aquifex aeolicus; Hydrogenobacter thermophilus; and Pyrococcus furiosus.

Hydrogenovibrio marinus hydrogenases have a relatively high catalytic activity and a high stability (thermal stability and oxygen resistance), and therefore their use provides various advantages. For example, Hydrogenovibrio marinus MH-110 deposited to the Institute of Physical and Chemical Research (Application reception number: 7688), which can be obtained relatively easily, may be used. Meanwhile, the Hydrogenovibrio marinus hydrogenase described in Japanese Patent Application Publication No. 2000-350585 (JP-A-2000-350585) may be used, for example. This Hydrogenovibrio marinu hydrogenase is constituted of small subunits having an amino acid sequence in the sequence number 2 and large subunits having an amino acid sequence in the sequence number 4. Meanwhile, several amino acids (e.g., one to three amino acid(s)) in the amino acid sequence of the hydrogenase may be subjected to various variations (e.g., removal, replacement, addition) including artificial ones.

Such hydrogenases origined from various microorganisms can be increased through cultivation of microorganisms. The culturing method may be decided according to the type of the microorganisms to be cultured. For example, Hydrogenovibrio marinus can be cultured by liquid culture or solid culture using a medium constituted of inorganic compounds under a gas phase of hydrogen, oxygen and carbon dioxide.

Hydrogenases are classified according to the types of their active sites. For example, there is a [Ni—Fe] type hydrogenase the active sites of which contain Ni—Fe and the electron-transfer sites of which contain Fe—S clusters. This [Ni—Fe] type hydrogenase is a heterodimer constituted of large subunits (active subunits) containing Ni—Fe and small subunits (electron-transfer subunits) containing three Fd—S clusters.

The modified hydrogenase of the invention can be obtained owing to the relatively low oxygen resistance of the electron-transfer subunits containing electron-transfer sites. More specifically, the intact-type hydrogenases are extracted from hydrogenase-producing bacteria and subjected to purification, the extracted intact-type hydrogenases are exposed to an oxygen atmosphere, whereby oxidative decomposition of the electron-transfer sites occurs and thus the electron-transfer sites are separated.

Other than membrane-bound type hydrogenases which are bound to cytoplasmic membranes, there are also known hydrogenases existing in periplasm and cytoplasm. In the case of the membrane-bound hydrogenases, when solubilizing membrane fractions, which are obtained by disrupting the hydrogenase-producing bacteria and then centrifuging them, the membrane fractions are exposed to an oxygen atmosphere and then, optionally, they are subjected to a solubilization process. Through these processes, the hydrogenases contained in the membrane fractions are exposed to the oxygen atmosphere and thus oxidized. As a result, the electron-transfer sites in the electron-transfer subunits constituting the hydrogenases are removed from the hydrogenases. At this time, usually, the whole electron-transfer subunits are removed from the hydrogenase. The conditions of exposure to the oxygen atmosphere differ depending upon the hydrogenase type, and therefore said conditions are set as needed within ranges in which a sufficient catalytic activity of the active sites can be obtained.

The methods for the above-described hydrogenase extraction from hydrogenase-producing bacteria and the purification are not specifically limited. That is, they may be typical methods. For example, in a case where hydrogenases are produced in the cells of the hydrogenase-producing bacteria, the bacterial cells are first suspended in a certain buffer solution, and then the bacterial cells are disrupted using a mechanical disruption method (e.g., a sonication method, a method using a French press, a method using a homogenizer with glass beads), a freezing-thawing method, and a frost-disruption method, whereby a disrupted liquid is obtained. The bacterial cells are removed from the liquid by centrifugal separation, and the membrane fractions are then conditioned. At this time, more specifically, a surfactant is added to the membrane fractions, whereby membrane proteins are solubilized. This process can be implemented using, for example, biochemical methods commonly used for purification of proteins, such as ammonium sulfate precipitation, gel-chromatography, ion-exchange chromatography, hydrophobic chromatography, affinity chromatography, etc. which may either be used alone or in combination.

On the other hand, in a case where hydrogenases are produced at the outsides of the bacterial cells, the culture fluid is used as it is or used after removal of the bacterial cells. Then, purification of hydrogenases is performed by a column chromatography including an ion-exchange chromatography and a gel filtration chromatography, which may either be used alone or in combination.

Before the above isolated-purification process by the column chromatography, a process for separating and purifying hydrogenases may be provided. This process is implemented to increase the purity of the hydrogenases in advance using, for example, salt precipitations using ammonium sulfate, etc., fractional precipitation using an organic solvent, denaturation precipitation by pH adjustment, iso-electric precipitation, and so on. Further, hydrogenases may be obtained as follows. First, hydrogenase genes are isolated from chromosome DNAs of microorganisms having hydrogenases, and then transformants having recombinant vectors containing the hydrogenase genes are then cultured, and hydrogenases are extracted from the obtained cultures.

As mentioned above, the modified hydrogenases of the invention have a high oxygen resistance and exhibit a stable catalytic activity under an oxygen atmosphere. Such modified hydrogenases having a stable catalytic activity can be used in various applications in various fields. For example, enzymatic electrodes that are utilized as electrode catalysts may be made of the modified hydrogenases. In this case, more specifically, the modified hydrogenases may be used as fuel electrodes of fuel cells that generate electric power through hydrogen oxidization (H₂→2H⁺+2e⁻) at an anode (fuel electrode) and oxygen reduction (4H⁺+O₂+4e⁻→2H₂O) at a cathode (oxidant electrode). Further, the modified hydrogenases may be applied to biosensors.

The forms of enzymatic electrodes for which the aforementioned modified hydrogenases are used are not specifically limited. For example, when the modified hydrogenases, which serve as electrode catalysts, are used, they may be fixed on a conductive substrate or may be dispersed in an electrolytic solution. The method for fixing the modified hydrogenases is not specifically limited. For example, it may be a method commonly used in the art. Usually, enzymes of hydrogenases are unable to perform efficient electron transfer with the conductive substrate, which is the electrode substrate. Therefore, electron transfer mediators may be used to mediate the electron transfer between the conductive substrate and the modified hydrogenases. As the electron transfer mediator, for example, methyl viologen or benzyl viologen may be used.

### First Example of Invention

### Conditioning of Modified Hydrogenases

First, 50 mM Tris-HCl buffer (pH 8.0) was added to the cells of Hydrogenovibrio marinus MH-110 and then suspended well. The amount of the added 50 mM Tris-HCl buffer was 5 ml per 1 g of bacterial cells (wet weight). Subsequently, a disruption process was performed three times using a sonication device (SONIFIER-450 (BRANSON)). The output level of the sonication device was 20 kHz, and the sonication duration was 2 minutes for each time. The sonicated liquid was then centrifuged at 8,000×g for 20 minutes at 4° C., and the supernatant of the centrifuged liquid was further ultracentrifuged at 128,000×g for 1 hour at 4° C., and cell membranes (membrane fractions) were obtained.

The obtained membrane fractions were then suspended using the same volume of a 50 mM Tris-HCl buffer (pH 8.0) containing 0.7 M ammonium sulfate. Subsequently, the membrane suspension was ultracentrifuged again for membrane washing. Then, a 50 mM Tris-HCI buffer (pH 8.0) containing 0.25% TritonX-100 and 10 mM EDTA were added to the membrane fractions. The amount of the added buffer was 10 times the amount of the bacterial cells (wet weight). Then, the liquid was solubilized by being stirred at 4° C. for 20 hours in air. Subsequently, the solubilized enzyme liquid was heated for 15 minutes after the heating temperature reached 55° C. and then cooled on an ice for an hour or longer. Then, the liquid was centrifuged at 20,000×g for 20 minutes at 4° C. for removing precipitations, and the supernatant of the liquid was then obtained as a solubilized liquid for the modified hydrogenases (electron-transfer-subunit free type hydrogenases).

Then, 0.7 M ammonium sulfate was added to the obtained solubilized liquid for the modified hydrogenases, and then the solubilized liquid was gently stirred. Subsequently, the conditioned liquid was centrifuged in the above-described manner and then subjected to Phenyl-Sepharose High Performance column chromatography. 20 mM Tris (pH 8.0) containing 0.7 M ammonium sulfate, 10% glycerol, and 1 μM n-hexadecyl-1-β-D-maltopyranoside were used as a purification buffer, and its concentration gradient was set so as to reduce the ammonium sulfate concentration, and thus the modified hydrogenases were fractionated.

Further, fractions exhibiting a benzylviologen (BV) reducing activity are subjected to Hydroxyapatite, and purification was performed at the concentration gradient of 1-400 mM potassium phosphate buffer (pH 6.8). A buffer containing 10% glycerol and 1 μMn-hexadecyl-1-β-D-maltopyranoside was used.

The obtained BV-active fractions were concentrated using AmiconUltra-15 and then subjected to gel-filtration using Superdex-200. 10 mM potassium phosphate buffer (pH 6.8) containing 100 mM ammonium sulfate, 10% glycerol and 1 μM n-hexadecyl-1-β-D-maltopyranoside were used as a buffer. This is how the modified hydrogenases were purified. The modified hydrogenases were dispensed to vial containers and stored in a gas phase of argon at a room temperature.

<Measurement of Activity of Modified Hydrogenases>

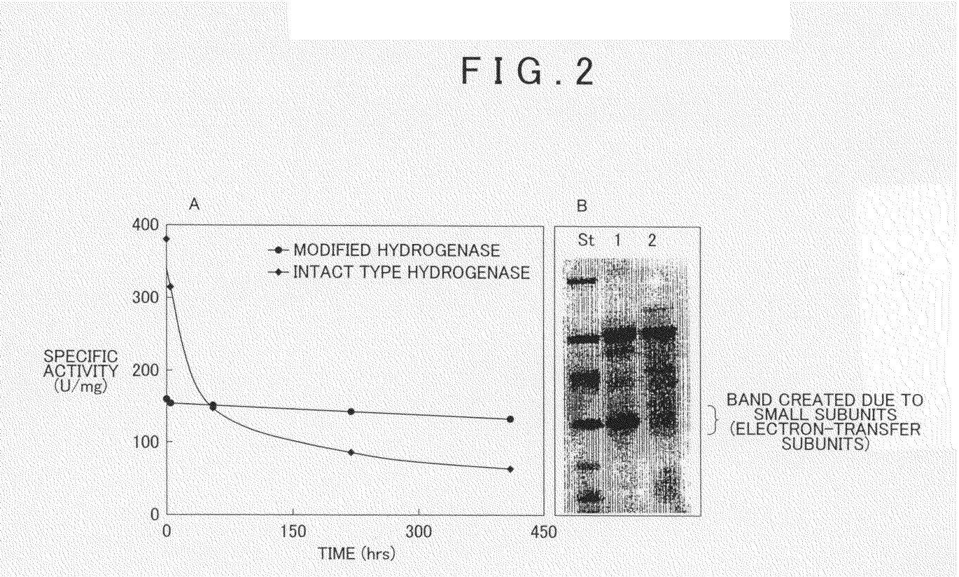

The activity of the obtained modified hydrogenases was measured taking as an one unit the amount of enzyme activity for reducing 1 μmol of benzylviologen in one minute in a hydrogen-saturated 50 mM potassium phosphate buffer (pH 7) at 60° C. FIG. 2A shows the result of this measurement.

<Electrophoresis of Modified Hydrogenases>

In the enzyme activity measurement described above, the modified hydrogenases were electrophoresed after being exposed to the atmosphere for 410 hours as follows. First, a denaturation buffer solution (60 mM Tris-HCl (pH 6.8) containing 2% sodium dodecyl sulfate (SDS), 20% glycerol, 10% 2-mercaptoethanol, 0.01% bromophenol blue) was mixed to the modified hydrogenases and denatured in a boiled water for 5 minutes. Then, it was subjected to SDS-polyacrylamide gel electrophoresis (SDS-PAGE). This electrophoresis was performed using the method of Laemmli (U.K. Laemmli, Nature 227: 680-685 (1970)). The separating gel concentration was 10%, and 25 mM Tris containing 0.192 M glycine, and 0.1% SDS were used as an electrophoresis buffer.

The result of the electrophoresis is shown in FIG. 2B. In FIG. 2B, “St” is a molecular-weight marker (standard specimen), “1” represents the result of electrophoresis of a first comparative example (intact type), and “2” represents the result of electrophoresis of the first example of the invention.

### First Comparative Example

### Purification of Intact Type, Hydrogenases

20 mM potassium phosphate buffer (pH 7.0) was added to the cells of Hydrogenovibrio marinus MH-110 and then suspended well. The amount of the added 20 mM potassium phosphate buffer was 5 ml per 1 g of the bacterial cells (wet weight). Subsequently, a disruption process was performed three times using a sonication devise (SONIFIER-450 (BRANSON)). The output level of the sonication device was 20 kHz, and the sonication duration was 2 minutes for each time. The disrupted (sonicated) liquid was then ultracentrifuged at 8,000×g for 20 minutes at 4° C., and the supernatant of the centrifuged liquid was further ultracentrifuged at 128,000×g for 1 hour at 4° C., after which the cell membranes (membrane fractions) were obtained.

Then, the obtained membrane fractions were suspended in a 20 mM potassium phosphate buffer (pH 7.0). 20 mM potassium phosphate buffer (pH 7.0) containing 1% TritonX-100 was mixed to the membrane fractions at the ratio of 1:1, so that the final concentration of TritonX-100 became 0.5%. Then, it was gently stirred for an hour at 4° C. under a gas phase of argon, whereby hydrogenases were solubilized. Then, a heating process was performed to denature and thus remove thermally unstable proteins. The heating process was performed for 15 minutes after the sample temperature reached 60° C., and it was then cooled on an ice for an hour or longer.

Next, the liquid was centrifuged at 20,000×g for 20 minutes at 4° C. for removing precipitations, and the supernatant of the liquid was then subjected to Q-Sepharose High-Performance column chromatography. 20MBis-Tris (pH 6.8), 10% glycerol, and 0.02% TritonX-100 were used as a purification buffer, and hydrogenases were obtained due to the gradient of the NaCl concentration. The active fractions obtained at the NaCl concentration of approx. 170 mM were subjected to column chromatography using Hydroxyapatite, whereby the active fractions were eluted by the concentration gradient of 1400 mM potassium phosphate buffer (pH 6.8). [0056] The hydrogenase active fractions eluted at the phosphate concentration of 45 to 55 mM were concentrated using Amicon Ultra-15 and then subjected to gel filtration of Superdex 200, so that they were purified up to a single band of electrophoresis. 10 mM potassium phosphate buffer (pH 6.8) containing 100 mM ammonium sulfate, 10% glycerol, and 0.02% TritonX-100 were used as a buffer for the gel filtration by Superdex-200.

The hydrogenases purified as above were dispensed to vial containers and stored in a gas phase of argon at a room temperature. Note that because the activity of the hydrogenases was stabilized due to the glycerol added, 10% glycerol was added to the buffers used in the respective purification processes. The buffer was anaerobically used by argon replacement.

<Measurement of Activity of Intact Type Hydrogenases>

The activity of the intact type hydrogenases was measured in the same manner as the modified hydrogenases of the first example of the invention. The result of this measurement is shown in FIG. 2A.

<Electrophoresis of Intact Type Hydrogenases>

The intact type hydrogenases were electrophoresed in the same manner as the modified hydrogenases of the first example of the invention. The result of this electrophoresis is shown in FIG. 2B.

(Results of Activity Measurement and Electrophoresis)

Referring to FIG. 2B, in the case of the intact type hydrogenases of the first comparative example, a dense band having a molecular weight of approx. 32,000 was created due to the small subunits (electron-transfer subunits). On the other band, in the case of the modified hydrogenases of the first example of the invention, almost no such small-subunit-derived bands were created, and this indicates that the small subunits were removed substantially completely. Further, with regard to the intact type hydrogenases of the first comparative example, referring to FIG. 2A, a sharp decrease in the activity of the hydrogenases was observed as a result of exposure to the atmosphere. This indicates that the electron transfer functions of the small subunits, which have a low oxygen resistance as mentioned above, weakened and thus their reactivity diminished. On the other hand, with regard to the modified hydrogenases of the first example of the invention, because the small subunits had been removed, even after exposed to the atmosphere for 410 hours, the modified hydrogenases had a sufficient direct electron-transfer reactivity from the activity center to benzylviologen, exhibiting a stable activity.

What is claimed is:

1. A modified hydrogenase obtained by removing electron-transfer sites from a hydrogenase constituted of: active subunits including active sites having a hydrogen oxidization-reduction activity; and electron-transfer subunits having the electron-transfer sites through which electrons are transferred between the active sites and the outside of the hydrogenase.

2. The modified hydrogenase according to claim 1, wherein the removal of the electron-transfer sites is accomplished by removing the electron-transfer subunits.

3. The modified hydrogenase according to claim 1, wherein the hydrogenase is a [Ni—Fe] hydrogenase constituted of active subunits having active sites containing Ni—Fe and electron-transfer subunits having electron-transfer sites containing Fe—S clusters.

4. A modified hydrogenate obtained from a hydrogenase constituted of: active subunits including active sites having a hydrogen oxidization-reduction activity; and electron-transfer subunits having electron-transfer sites through which electrons are transferred between the active sites and the outside of the hydrogenase, wherein
when the hydrogenase is isolated from bacteria that produces the hydrogenase, a process for exposing the hydrogenase to an oxygen atmosphere is executed.

5. The modified hydrogenase according to claim 4, wherein the process for exposing the hydrogenase to the oxygen atmosphere is a process in which membrane fractions are isolated from the hydrogenase-producing bacteria, the isolated membrane fractions are exposed to an oxygen atmosphere, and then the hydrogenase is solubilized from the membrane fractions.

6. The modified hydrogenase according to claim 4, wherein the process for exposing the hydrogenase to the oxygen atmosphere is a process in which membrane fractions are isolated from the hydrogenase-producing bacteria and the hydrogenase is solubilized from the membrane fractions under an oxygen atmosphere.

7. The modified hydrogenase according to claim 4, wherein the hydrogenase is a [Ni—Fe] hydrogenase constituted of active subunits having active sites containing Ni—Fe and electron-transfer subunits having electron-transfer sites containing Fe—S clusters.

8. The modified hydrogenase according to claim 1, wherein the hydrogenase is a hydrogenase originated from Hydrogenovibrio marinus.

9. The modified hydrogenase according to claim 4, wherein the hydrogenase is a hydrogenase originated from Hydrogenovibrio marinus.

10. An enzymatic electrode made of the modified hydrogenase according to claim 1.

11. An enzymatic electrode made of the modified hydrogenase according to claim 4.

12. An enzymatic electrode according to claim 10, wherein the enzymatic electrode is used in a fuel cell and the modified hydrogenase is used as an electrode catalyst.

13. A hydrogenase modification method, comprising:
isolating from hydrogenase-producing bacteria a hydrogenase constituted of: active subunits including active sites having a hydrogen oxidization-reduction activity; and electron-transfer subunits having electron-transfer sites through which electrons are transferred between the active sites and the outside of the hydrogenase; and
removing the electron-transfer sites of the electron-transfer subunits from the hydrogenase by exposing the hydrogenase to an oxygen atmosphere.

14. The hydrogenase modification method according to claim 13, wherein
the isolation of the hydrogenase is accomplished by isolating membrane fractions from the hydrogenase-producing bacteria, and
the exposure of the hydrogenase to the oxygen atmosphere is accomplished by exposing the isolated membrane fractions to the oxygen atmosphere and then solubilizing the hydrogenase from the membrane fractions.

15. The hydrogenase modification method according to claim 13, wherein
the isolation of the hydrogenase is accomplished by isolating membrane fractions from the hydrogenase-producing bacteria, and
the exposure of the hydrogenase to the oxygen atmosphere is accomplished by solubilizing the hydrogenase from the isolated membrane fractions under an oxygen atmosphere.
